SEQUENCE LISTING (23 sequences)

SEQ ID NO: 1 - protein, 6 aa, Artificial
  Chemically synthesized
FCIGRL

SEQ ID NO: 2 - protein, 6 aa, Artificial
  Chemically synthesized
  misc_feature (1)..(1): Xaa can be Ala, Val, Leu, Ile, Pro, Trp, Tyr, and Met
XCIGRL

SEQ ID NO: 3 - protein, 6 aa, Artificial
  Chemically synthesized
  misc_feature (2)..(2): Xaa can be Gly, Ser, Thr, Tyr, Asn, and Gln
FXIGRL

SEQ ID NO: 4 - protein, 6 aa, Artificial
  Chemically synthesized
  misc_feature (3)..(3): Xaa can be Ala, Val, Leu, Ile, Pro, Trp, and Met
FCXGRL

SEQ ID NO: 5 - protein, 6 aa, Artificial
  Chemically synthesized
  misc_feature (4)..(4): Xaa can be Gly, Ser, Thr, Tyr, Asn, Ala, and Gln
FCIXRL

SEQ ID NO: 6 - protein, 6 aa, Artificial
  Chemically synthesized
  misc_feature (5)..(5): Xaa can be Lys and His
FCIGXL

SEQ ID NO: 7 - protein, 6 aa, Artificial
  Chemically synthesized
  misc_feature (6)..(6): Xaa can be Ala, Val, Leu, Ile, Pro, Trp, and Met
FCIGRX

SEQ ID NO: 8 - protein, 6 aa, Artificial
  Chemically synthesized
  misc_feature (1)..(1): Xaa can be Ala, Val, Leu, Ile, Pro, Trp, Tyr, and Met
  misc_feature (2)..(2): Xaa can be Gly, Ser, Thr, Tyr, Asn, and Gln
XXIGRL

SEQ ID NO: 9 - protein, 6 aa, Artificial
  Chemically synthesized
  misc_feature (1)..(1): Xaa can be Ala, Val, Leu, Ile, Pro, Trp, Tyr, and Met
  misc_feature (3)..(3): Xaa can be Ala, Val, Leu, Ile, Pro, Trp, and Met
XCXGRL

SEQ ID NO: 10 - protein, 6 aa, Artificial
  Chemically synthesized
  misc_feature (1)..(1): Xaa can be Ala, Val, Leu, Ile, Pro, Trp, Tyr, and Met
  misc_feature (4)..(4): Xaa can be Gly, Ser, Thr, Tyr, Asn, Ala, and Gln
XCIXRL

SEQ ID NO: 11 - protein, 6 aa, Artificial
  Chemically synthesized
  misc_feature (1)..(1): Xaa can be Ala, Val, Leu, Ile, Pro, Trp, Tyr, and Met
  misc_feature (5)..(5): Xaa can be Lys and His
XCIGXL

SEQ ID NO: 12 - protein, 6 aa, Artificial
  Chemically synthesized
  misc_feature (1)..(1): Xaa can be Ala, Val, Leu, Ile, Pro, Trp, Tyr, and Met
  misc_feature (6)..(6): Xaa can be Ala, Val, Leu, Ile, Pro, Trp, and Met
XCIGRX

SEQ ID NO: 13 - protein, 6 aa, Artificial
  Chemically synthesized
  misc_feature (2)..(2): Xaa can be Gly, Ser, Thr, Tyr, Asn, and Gln
  misc_feature (3)..(3): Xaa can be Ala, Val, Leu, Ile, Pro, Trp, and Met
FXXGRL

SEQ ID NO: 14 - protein, 6 aa, Artificial
  Chemically synthesized
  misc_feature (2)..(2): Xaa can be Gly, Ser, Thr, Tyr, Asn, and Gln
  misc_feature (4)..(4): Xaa can be Gly, Ser, Thr, Tyr, Asn, Ala, and Gln
FXIXRL

SEQ ID NO: 15 - protein, 6 aa, Artificial
  Chemically synthesized
  misc_feature (2)..(2): Xaa can be Gly, Ser, Thr, Tyr, Asn, and Gln
  misc_feature (5)..(5): Xaa can be Lys and His
FXIGXL

SEQ ID NO: 16 - protein, 6 aa, Artificial
  Chemically synthesized
  misc_feature (2)..(2): Xaa can be Gly, Ser, Thr, Tyr, Asn, and Gln
  misc_feature (6)..(6): Xaa can be Ala, Val, Leu, Ile, Pro, Trp, and Met
FXIGRX

SEQ ID NO: 17 - protein, 6 aa, Artificial
  Chemically synthesized
  misc_feature (3)..(3): Xaa can be Ala, Val, Leu, Ile, Pro, Trp, and Met
  misc_feature (4)..(4): Xaa can be Gly, Ser, Thr, Tyr, Asn, Ala, and Gln
FCXXRL

SEQ ID NO: 18 - protein, 6 aa, Artificial
  Chemically synthesized
  misc_feature (3)..(3): Xaa can be Ala, Val, Leu, Ile, Pro, Trp, and Met
  misc_feature (5)..(5): Xaa can be Lys and His
FCXGXL

SEQ ID NO: 19 - protein, 6 aa, Artificial
  Chemically synthesized
  misc_feature (3)..(3): Xaa can be Ala, Val, Leu, Ile, Pro, Trp, and Met
  misc_feature (6)..(6): Xaa can be Ala, Val, Leu, Ile, Pro, Trp, and Met
FCXGRX

SEQ ID NO: 20 - protein, 6 aa, Artificial
  Chemically synthesized
  misc_feature (4)..(4): Xaa can be Gly, Ser, Thr, Tyr, Asn, Ala, and Gln
  misc_feature (5)..(5): Xaa can be Lys and His
FCIXXL

SEQ ID NO: 21 - protein, 6 aa, Artificial
  Chemically synthesized
  misc_feature (4)..(4): Xaa can be Gly, Ser, Thr, Tyr, Asn, Ala, and Gln
  misc_feature (6)..(6): Xaa can be Ala, Val, Leu, Ile, Pro, Trp, and Met
FCIXRX

SEQ ID NO: 22 - protein, 6 aa, Artificial
  Chemically synthesized
  misc_feature (5)..(5): Xaa can be Lys and His
  misc_feature (6)..(6): Xaa can be Ala, Val, Leu, Ile, Pro, Trp, and Met
FCIGXX

SEQ ID NO: 23 - protein, 399 aa, Vibrio cholerae
MSIFIHHGAPGSYKTSGALWLRLLPAIKSGRHIITNVRGLNLERMAKYLKMDVSDISIEFIDTDHPDGRLTMARFWHWARKDAFLFIDECGRIWPPRLTATNLKALDTPPDLVAEDRPESFEVAFDMHRHHGWDICLTTPNIAKVHNMIREAAEIGYRHFNRATVGLGAKFTLTTHDAANSGQMDSHALTRQVKKIPSPIFKMYASTTTGKARDTMAGTALWKDRKILFLFGMVFLMFSYSFYGLHDNPIFTGGNDATIESEQSEPQSKATAGNAVGSKAVAPASFGFCIGRLCVQDGFVTVGDERYRLVDNLDIPYRGLWATGHHIYKDKLTVFFETESGSVPTELFASSYRYKVLPLPDFNHFVVFDTFAAQALWVEVKRGLPIKTENDKKGLNSIF

(19) United States
(12) Patent Application Publication
(10) Pub. No.: US 2007/0196272 A1
(43) Pub. Date: Aug. 23, 2007

(54) ORAL DELIVERY OF THERAPEUTIC AGENTS USING TIGHT JUNCTION AGONISTS

(75) Inventors: Natalie D. Eddington, Baltimore, MD (US); Alessio Fasano, West Friendship, MD (US); Keon-Hyoung Song, Ellicott City, MD (US)

Correspondence Address: Connolly Bove Lodge Hutz, LLP;(FOR ALBA THERAPEUTICS), P.O. BOX 2207, WILMINGTON, DE, 19899-2207, US

(73) Assignee: UNIVERSITY OF MARYLAND, BALTIMORE, Baltimore, MD (US)

(21) Appl. No.: 11/673,192

(22) Filed: Feb. 9, 2007

Related U.S. Application Data
(60) Provisional application No. 60/771,453, filed on Feb. 9, 2006.

(51) Int. Cl. A61K 51/00; A61K 38/28; A61K 38/14; A61K 38/22; A61K 31/727; A61K 38/48; A61K 31/56; A61M 36/14

(52) U.S. Cl. 424/1.11; 514/14; 514/15; 514/16; 514/17; 514/8; 514/12; 514/3; 514/11; 514/171; 514/56; 514/44; 424/94.63; 514/18

(57) ABSTRACT

The present invention provides compositions and methods for the administration of the compositions to mammals. The compositions comprise therapeutic agents and an intestinal absorption enhancing amount of one or more tight junction agonists. Tight junction agonists include zonulin and/or ZOT receptor agonists. Methods of the invention include orally administering compositions of the invention.

### CROSS REFERENCE TO RELATED APPLICATIONS

This application claims priority to U.S. provisional patent application Ser. No. 60/771,453, filed Feb. 9, 2006, the entire contents of which are specifically incorporated herein by reference.

### STATEMENT REGARDING FEDERALLY SPONSORED RESEARCH

This research was funded by NIH Grant 2-R01 EB02771 and MH067507. The United States government may have certain rights in the invention.

### BACKGROUND OF THE INVENTION

The low bioavailability (BA) of efficacious pharmacotherapeutic drugs continues to be a major obstacle in drug development and in many instances may be the deciding factor on whether or not a potent agent is developed. These therapeutic agents experience low BA after oral administration due to poor absorption or susceptibility to first pass metabolism. The search for an efficient novel drug delivery system to overcome this problem cannot be overemphasized. A means of enhancing the gastrointestinal absorption of these drugs would significantly extend their therapeutic usefulness as well as decreasing the dose required to produce efficacy.

Absorption enhancers, including surfactants, fatty acids, and chitosan derivatives, have been used to modify bioavailability by either disruption of the cell membrane or modulation of the tight junctions (TJ) (1). In general, the optimal absorption enhancer should possess the following qualities; its effect should be reversible, it should provide a rapid permeation enhancing effect on the intestinal cellular membrane, it should be non-cytotoxic at the effective concentration level without deleterious and/or irreversible effects on the cellular membrane or cytoskeleton of the TJ. Zonula Occludens Toxin (ZOT), a 44.8 kDa protein (399 amino acids; AA) located in the cell envelope of the bacterial strain Vibrio cholerae, is capable of reversibly opening the TJ between cells and increasing the paracellular transport of many drugs in a non-toxic manner (2-7). Intensive investigation of the biological activity of ZOT as an absorption enhancer was triggered by reports of effective oral administration of insulin with ZOT in diabetic rats (4). Recently, a smaller 12 kDa fragment (AA 265-399) of ZOT, referred to as delta G (ΔG), was introduced as the biologically active fragment of ZOT (8). Amino acid comparison between ZOT active fragment and Zonulin, combined with site-directed mutagenesis experiments, confirmed the presence of an octapeptide receptor-binding domain toward the amino terminus of the processed ZOT.

### SUMMARY OF THE INVENTION

The methods and compositions of the invention relate broadly to methods and compositions for enhancing absorption of a therapeutic agent by mucosal tissues. Thus, the composition can be administered to a subject by any suitable route, including orally. In one aspect, the composition is directly or indirectly administered to the gut. For example, the methods and compositions of the invention are useful for enhancing absorption in the intestine, including the duodenum, jejunum, ileum, and colon. More particularly, in one aspect the invention is drawn to enhancing absorption in the small intestine.

In one aspect, the invention comprises a therapeutic composition comprising a therapeutically effective amount of one or more therapeutic agents and an intestinal absorption enhancing amount of one or more tight junction agonists, for example zonulin and/or ZOT receptor agonists. A zonulin and/or ZOT receptor agonist is a compound which is believed to mediate tight junction opening through the same receptor utilized by zonula occludens toxin (ZOT). In a particular aspect, the invention comprises a composition wherein at least one of the one or more zonulin and/or ZOT receptor agonists comprises a peptide. The peptide can comprise from about 6 to about 50 amino acid residues. In another aspect, the peptide can comprise from about 6 to about 25 amino acid residues. In yet another aspect, the peptide can comprise from about 6 to about 15 amino acid residues. In another aspect the peptide may be from about 6 to about 9 amino acids. In one particular aspect, the peptide can comprise a sequence selected from the group consisting of FCIGRX, FCIGXL, FCIXRL, FCXGRL, FXIGRL, XCIGRL, XXIGRL, XCXGRL, XCIXRL, XCIGXL, XCIGRX, FXXGRL, FXIXRL, FXIGXL, FXIGRX, FCXXRL, FCXGXL, FCXGRX, FCIXXL, FCIXRX, and FCIGXX, wherein each X is independently a natural or synthetic amino acid residue. Moreover, the invention can comprise a composition wherein at least one of the one or more zonulin and/or ZOT receptor agonists is a peptide comprising the sequence FCIGRL (SEQ ID NO:1). Indeed the peptide can be H—FCIGRL-OH.

In another aspect, the invention comprises a composition wherein at least one therapeutic agent is selected from the group consisting of an antibiotic, an anti-inflammatory, an analgesic, an immunosuppressant, and a peptide hormone.

The composition of the invention can comprise a peptide hormone which can be insulin.

The composition of the invention can also comprise one or more therapeutic agents wherein at least one of the one or more therapeutic agents is selected from the group consisting of a small molecule, a peptide, a protein, a lipid, a carbohydrate, and combinations thereof.

In one aspect, the composition is in aqueous solution.

In another aspect, the composition further comprises one or more protease inhibitors.

The composition can further comprise one or more pharmaceutically acceptable excipients.

In still another aspect, the invention comprises a composition wherein at least one of the one or more tight junction agonists (e.g., zonulin and/or ZOT receptor agonists) is a peptide comprising the sequence FCIGRL and the composition further comprises at least one protease inhibitor and one or more therapeutic agents selected from the group consisting of a small molecule, a peptide, a protein, a lipid, and a carbohydrate, and combinations thereof.

In another aspect, the invention comprises a method of treating a subject comprising orally administering to the subject the composition of the invention. In a particular aspect, the composition can comprise one or more therapeutic agents and an intestinal absorption enhancing amount of one or more tight junction agonists (e.g., zonulin and/or ZOT receptor agonists). The subject can be a mammal. In one particular aspect, the subject is a human.

In yet another aspect, the invention comprises a method of treating diabetes in an animal in need thereof, comprising: orally administering to the animal a composition comprising an insulin, a derivative of an insulin, or a combination thereof, and an intestinal absorption enhancing amount of one or more tight junction agonists (e.g., zonulin and/or ZOT receptor agonists).

### BRIEF DESCRIPTION OF THE FIGURES

FIG. 1. Amino acid sequence of ZOT (SEQ ID NO: 23). Highlighted (265-399) is delta G, the biologically active fragment of ZOT, and box (288-293) is AT1002, active domain of ZOT.

FIG. 2. Average plasma concentration versus time profile for CsA in jugular cannulated Sprague-Dawley rats following the ID administration of four treatments. i.e., CsA (●), CsA/AT1002 (◯), CsA/PI/BC (▾), and CsA/PI/BC/AT1002 (CsA 120 μCi/kg, PI (bestatin 30 mg/kg and E-64 10 mg/kg), BC 0.1 w/v %, and/or AT1002 5 (∇), 10(▪) or 40 mg/kg (□)). Each data point represents the mean±SEM of 4-5 rats. * Significant at p<0.05 compared to CsA/PI/BC of each same time point, ** Significant at p<0.05 compared to CsA/PI/BC+AT1002 5 mg/kg of same time point.

FIG. 3. Average plasma concentration of CsA versus AT1002 dose profile in jugular cannulated Sprague-Dawley rats following the ID administration of each dose of AT1002 (0, 5, 10, and 40 mg/kg) with CsA/PI/BC (CsA 120 μCi/kg, PI (bestatin 30 mg/kg and E-64 10 mg/kg) and BC 0.1 w/v %, respectively). Each bar is expressed as the mean±SEM for 4-5 rats. * Significant p<0.05 compared to CsA/PI/BC of each same time point.

### DETAILED DESCRIPTION

The present invention provides for the enhanced uptake of compositions (e.g., therapeutic compositions) from mucosal surfaces using one or more tight junction agonist. An example of a tight junction agonist is zonula occludens toxin (ZOT), which is produced by Vibrio cholerae. A ZOT receptor agonist is a compound which is believed to mediate tight junction opening through the same receptor utilized by ZOT. In another embodiment, a tight junction agonist may comprise zonulin. A zonulin receptor agonist is a compound which is believed to mediate tight junction opening through the same receptor utilized by zonulin. Both ZOT receptor agonists and zonulin receptor agonists are examples of tight junction agonists. Without wishing to be bound by theory, it is believed that ZOT and zonulin utilize the same receptor while functioning as tight junction agonists. Zonula Occludens Toxin (ZOT) and its biologically active fragment, Delta G have been shown to reversibly open tight junctions (TJ) in endothelial and epithelial cells. Recently, a six-mer synthetic peptide H—FCIGRL-OH (AT1002) was identified and synthesized that retains the ZOT permeating effect on intercellular TJ. The objective of this study was to evaluate the biological activity of AT1002 on enhancing the oral administration of Cyclosporine A (CsA).

The present invention also contemplates the use of functional derivatives of AT1002. Examples include, but are not limited to,

| | |
|---|---|
| Xaa1 Cys Ile Gly Arg Leu, | (SEQ ID NO: 2) |
| Phe Xaa2 Ile Gly Arg Leu, | (SEQ ID NO: 3) |
| Phe Cys Xaa3 Gly Arg Leu, | (SEQ ID NO: 4) |
| Phe Cys Ile Xaa4 Arg Leu, | (SEQ ID NO: 5) |
| Phe Cys Ile Gly Xaa5 Leu, | (SEQ ID NO: 6) |
| and | |
| Phe Cys Ile Gly Arg Xaa6. | (SEQ ID NO: 7) |

Xaa1 may be selected from the group consisting of Ala, Val, Leu, Ile, Pro, Trp, Tyr, and Met; Xaa2 may be selected from the group consisting of Gly, Ser, Thr, Tyr, Asn, and Gln; Xaa3 may be selected from the group consisting of Ala, Val, Leu, Ile, Pro, Trp, and Met; Xaa4 may be selected from the group consisting of Gly, Ser, Thr, Tyr, Asn, Ala, and Gln; Xaa5 may be selected from the group consisting of Lys and His; and Xaa6 may be selected from the group consisting of Ala, Val, Leu, Ile, Pro, Trp, and Met.

Further functional derivatives of (SEQ ID NO:1) include:

| | |
|---|---|
| Xaa1 Xaa2 Ile Gly Arg Leu, | (SEQ ID NO: 8) |
| Xaa1 Cys Xaa3 Gly Arg Leu, | (SEQ ID NO: 9) |
| Xaa1 Cys Ile Xaa4 Arg Leu, | (SEQ ID NO: 10) |
| Xaa1 Cys Ile Gly Xaa5 Leu, | (SEQ ID NO: 11) |
| Xaa1 Cys Ile Gly Arg Xaa6, | (SEQ ID NO: 12) |
| Phe Xaa2 Xaa3 Gly Arg Leu, | (SEQ ID NO: 13) |
| Phe Xaa2 Ile Xaa4 Arg Leu, | (SEQ ID NO: 14) |
| Phe Xaa2 Ile Gly Xaa5 Leu, | (SEQ ID NO: 15) |
| Phe Xaa2 Ile Gly Arg Xaa6, | (SEQ ID NO: 16) |
| Phe Cys Xaa3 Xaa4 Arg Leu, | (SEQ ID NO: 17) |
| Phe Cys Xaa3 Gly Xaa5 Leu, | (SEQ ID NO: 18) |
| Phe Cys Xaa3 Gly Arg Xaa6, | (SEQ ID NO: 19) |
| Phe Cys Ile Xaa4 Xaa5 Leu, | (SEQ ID NO: 20) |
| Phe Cys Ile Xaa4 Arg Xaa6, | (SEQ ID NO: 21) |
| and | |
| Phe Cys Ile Gly Xaa5 Xaa6. | (SEQ ID NO: 22) |

Xaa1 may be selected from the group consisting of Ala, Val, Leu, Ile, Pro, Trp, Tyr, and Met; Xaa2 is selected from the group consisting of Gly, Ser, Thr, Tyr, Asn, and Gln; Xaa3 is selected from the group consisting of Ala, Val, Leu, Ile, Pro, Trp, and Met; Xaa4 is selected from the group consisting of Gly, Ser, Thr, Tyr, Asn, Ala, and Gln; Xaa5 is selected from the group consisting of Lys and His; Xaa6 is selected from the group consisting of Ala, Val, Leu, Ile, Pro, Trp, and Met.

When the tight junction agonist is a peptide, any length of peptide may be used. For example, an agonist may be about 3, about 4, about 5, about 6, about 7, about 8, about 9, about 10, about 11, about 12, about 13, about 14 or about 15 amino acids in length. In some embodiments, a peptide tight junction agonist may be from about 3 to about 12, from about 4 to about 12, from about 5 to about 12, from about 6 to about 12, from about 7 to about 12, from about 8 to about 12, from about 9 to about 12, from about 10 to about 12, from about 3 to about 10, from about 4 to about 10, from about 5 to about 10, from about 6 to about 10, from about 7 to about 10, from about 8 to about 10, from about 9 to about 10 amino acids in length. In some embodiments, a peptide tight junction agonist may be 9 amino acids or less in length.

The intestinal permeability enhancing effect of AT1002 on the transport of CsA across Caco-2 cell monolayers was examined after the following treatments, i.e., CsA, CsA/protease inhibitors(PI), CsA/PI/benzalkonium chloride(BC), CsA/AT1002, CsA/PI/AT1002, and CsA/PI/BC/AT1002 (CsA 0.5 μCi/ml, PI (bestatin 15 mM and E64 5 mM), BC 0.005 w/v %, and AT1002 5 mM, respectively). Apparent permeability coefficients (Pₐₚₚ) were calculated for each treatment. In addition, four treatments, i.e., CsA, CsA/PI/BC, CsA/AT1002, and CsA/PI/BC/AT1002 (CsA 120 μCi/kg, PI (bestatin 30 mg/kg and E-64 10 mg/kg), BC 0.1% w/v, and AT1002 doses of 5, 10 or 40 mg/kg, respectively) were prepared and administered intraduodenally to male Sprague-Dawley rats (230-280 g, n=4-5). Blood samples were collected at 0, 20, 60, and 120 min post-dosing and CsA plasma concentrations were determined subsequently using a Beckman Liquid Scintillation Counter.

No significant increases in CsA transport were observed in the Caco-2 cell culture experiments following pre-treatment with AT1002 (5 mM). However, AT1002 appeared to increase the Pₐₚₚ of CsA by 120% (1.54±0.13×10⁻⁶ cm/sec) and 111% (1.76±0.05×10⁻⁶ cm/sec) in each treatment (CsA/AT1002 and CsA/PI/AT1002) compared to each control (CsA and CsA/PI) respectively. The plasma concentration of CsA was significantly increased over a range of 155% to 250% at 10 mg/kg and 40 mg/kg dose of AT1002. Also, AUC_{0-120 min} of CsA over a range of 164% to 214% and the Cₘₐₓ of CsA over a range of 177% to 256% was statistically and significantly increased at 10 mg/kg and 40 mg/kg of AT1002 after the intraduodenal administration of CsA/PI/BC/AT1002 to Sprague-Dawley rats.

AT1002 significantly increased the in vivo oral absorption of CsA in the presence of PI. This study demonstrates that AT1002-mediated tight junction modulation, combined with metabolic protection and stabilization, may be used to enhance the low oral bioavailability of certain drugs when administered concurrently.

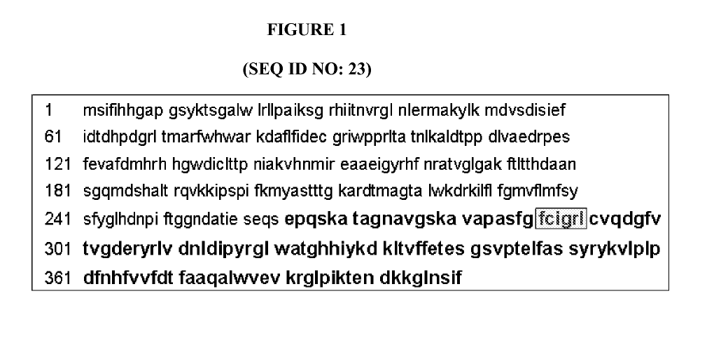

Studies in our laboratory have shown that ZOT enhances the intestinal transport of drug candidates of varying molecular weight (mannitol, PEG4000, Inulin, and sucrose) or low BA (paclitaxel, acyclovir, cyclosporin A, and doxorubicin) across Caco-2 cell monolayers (6,7) and the transport enhancing effect of ZOT is reversible and non-toxic (2,7). In addition, ΔG significantly increased the in vitro transport of paracellular markers (mannitol, PEG4000, and Inulin) in a nontoxic manner and the in vivo absorption of low bioavailable therapeutic agents (cyclosporin A, ritonavir, saquinavir, and acyclovir) (9-11). Even though promising results were obtained with the use of ΔG with therapeutic agents, the isolation/purification process did not yield sufficient amounts of biologically active ΔG to allow for conduct of in vivo studies at higher dose. In an attempt to resolve this issue, several modifications of ΔG sequence were evaluated (8). It was noted that the amino acid sequence ‘IGRL’, identified as part of the binding domain in ZOT/delta G is the same as that observed in the PAR-2 agonists (fur-LIGRL, FCIGRL) (8). PAR-2 agonists have been reported to increase paracellular permeability (ref). As such, this lead to the hypothesis that ZOT/ΔG may act at these receptors and produce an increase in the paracellular permeability. Recently, AT1002, a six-mer synthetic peptide H—FCIGRL-OH, was isolated from the active fragment of ΔG, subsequently synthesized and assumed to retain ΔG or ZOT permeating effect on intercellular TJ. FCIGRL is identical to the AA residues 288-293 of ZOT and the XX—IGRL sequence is part of the putative receptor binding motif of ZOT/ΔG, thus the peptide was expected to have similar properties as ZOT/ΔG (FIG. 1). Hence, this is the first study to evaluate the effectiveness of AT1002 as an absorption enhancer after oral co-administration with a low bioavailable therapeutic agent. Cyclosporin A (CsA) as a low bioavailable therapeutic agent is a potent immunosuppressant agent with high molecular weight, efflux properties, and low oral BA (<20%) (12). Increases in the absorption of CsA would suggest that AT1002 could be used to improve the BA for novel therapeutic macromolecules (e.g., proteins, peptides, and peptidomimetics).

The immunosuppressant used in the method and composition of the invention can be any agent which tends to attenuate the activity of the humoral or cellular immune systems. In particular, the invention comprises a composition wherein the immunosuppressant is selected from the group consisting of cyclosporin A, FK506, prednisone, methylprednisolone, cyclophosphamide, thalidomide, azathioprine, and daclizumab, physalin B, physalin F, physalin G, seco-steroids purified from Physalis angulata L., DSG(15-deoxyspergualin, 15-dos), MMF, rapamycin and its derivatives, CCI-779, FR 900520, FR 900523, NK86-1086, depsidomycin, kanglemycin-C, spergualin, prodigiosin25-c, cammunomicin, demethomycin, tetranactln, tranilast, stevastelins, myriocin, gllooxin, FR 651814, SDZ214-104, bredinin, WS9482, mycophenolic acid, 15-deoxyspergualin, mimoribine, misoprostol, OKT3, anti-IL-2 receptor antibodies, azasporine, leflunomide, mizoribine, azaspirane (SKF 105685), paclitaxel, altretamine, busulfan, chlorambucil, ifosfamide, mechlorethamine, melphalan, thiotepa, cladribine, fluorouracil, floxuridine, gemcitabine, thioguanine, pentostatin, methotrexate, 6-mercaptopurine, cytarabine, carmustine, lomustine, streptozotocin, carboplatin, cisplatin, oxaliplatin, iproplatin, tetraplatin, lobaplatin, JM216, JM335, fludarabine, aminoglutethimide, flutamide, goserelin, leuprolide, megestrol acetate, cyproterone acetate, tamoxifen, anastrozole, bicalutamide, dexamethasone, diethylstilbestrol, bleomycin, dactinomycin, daunorubicin, doxirubicin, idarubicin, mitoxantrone, losoxantrone, mitomycin-c, plicamycin, paclitaxel, docetaxel, topotecan, irinotecan, 9-amino camptothecan, 9-nitro camptothecan, GS-211, etoposide, teniposide, vinblastine, vincristine, vinorelbine, procarbazine, asparaginase, pegaspargase, octreotide, estramustine, and hydroxyurea, and combinations thereof. In one more particular aspect, the immunosuppressant is cyclosporin A.

Furthermore, the therapeutic agent can be selected from the group consisting of a chemotherapeutic, a gene therapy vector, a growth factor, parathyroid hormone, human growth hormone, a contrast agent, an angiogenesis factor, a radionuclide, an anti-infection agent, an anti-tumor compound, a receptor-bound agent, a hormone, a steroid, a protein, a complexing agent, a polymer, heparin, covalent heparin, a thrombin inhibitor, hirudin, hirulog, argatroban, D-phenylalanyl-L-poly-L-arginyl chloromethyl ketone, an antithrombogenic agent, urokinase, streptokinase, a tissue plasminogen activator, a thrombolytic agent, a fibrinolytic agent, a vasospasm inhibitor, a calcium channel blocker, a nitrate, nitric oxide, a nitric oxide promoter, a vasodilator, an antihypertensive agent, an antimicrobial agent, an antibiotic, aspirin, triclopidine, a glycoprotein IIb/IIIa inhibitor, an inhibitor of surface glycoprotein receptors, an antiplatelet agent, colchicine, an antimitotic, a microtubule inhibitor, dimethyl sulfoxide (DMSO), a retinoid, an antisecretory agent, cytochalasin, an actin inhibitor, a remodeling inhibitor, deoxyribonucleic acid, an antisense nucleotide, an agent for molecular genetic intervention, methotrexate, an antimetabolite, an antiproliferative agent, tamoxifen citrate, an anti-cancer agent, dexamethasone, dexamethasone sodium phosphate, dexamethasone acetate, a dexamethasone derivative, an anti-inflammatory steroid, a non-steroidal antiinflammatory agent, cyclosporin, an immunosuppressive agent, trapidal, a PDGF antagonist, angiopeptin, a growth hormone antagonist, angiogenin, a growth factor antibody, an anti-growth factor antibody, a growth factor antagonist, dopamine, bromocriptine mesylate, pergolide mesylate, a dopamine agonist, ⁶⁰Co, ¹⁹²Ir, ³²P, ¹¹¹In, ⁹⁰Y, ⁹⁹mTc, a radiotherapeutic agent, an iodine-containing compound, a barium-containing compound, gold, tantalum, platinum, tungsten, a heavy metal functioning as a radiopaque agent, a peptide, a protein, an enzyme, an extracellular matrix component, a cellular component, captopril, enalapril, an angiotensin converting enzyme (ACE) inhibitor, ascorbic acid, α-tocopherol, superoxide dismutase, deferoxamine, a 21-aminosteroid (lasaroid), a free radical scavenger, an iron chelator, an antioxidant, a ¹⁴C—, ³H—, ¹³¹I—, ³²P— or ³⁶S-radiolabelled form or other radiolabelled form of any of the foregoing, estrogen, a sex hormone, AZT, an antipolymerases, acyclovir, famciclovir, rimantadine hydrochloride, ganciclovir sodium, an antiviral agents, 5-aminolevulinic acid, meta-tetrahydroxyphenylchlorin, hexadecafluoro zinc phthalocyanine, tetramethyl hematoporphyrin, rhodamine 123 or other photodynamic therapy agents, an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents, gene therapy agents, enalapril, a prodrug, and an agent for treating benign prostatic hyperplasia (BHP), or combinations thereof.

The composition can further comprise one or more protease inhibitors. Any protease inhibitor can be used, including, but not limited to, a proteinase, peptidase, endopeptidase, or exopeptidase inhibitor. Certainly a cocktail of inhibitors can also be used, if appropriate. Alternatively, the protease inhibitors can be selected from the group consisting of bestatin, L-trans-3-carboxyoxiran-2-carbonyl-L-leucylagmatine, ethylenediaminetetraacetic acid (EDTA), phenylmethylsulfonylfluoride (PMSF), aprotinin, amyloid protein precursor (APP), amyloid beta precursor protein, α1-proteinase inhibitor, collagen VI, bovine pancreatic trypsin inhibitor (BPTI), 4-(2-aminoethyl)-benzenesulfonyl fluoride (AEBSF), antipain, benzamidine, chymostatin, ε-aminocaproate, N-ethylmaleimide, leupeptin, pepstatin A, phosphoramidon, and combinations thereof. Novel protease inhibitors can also be used. Indeed, protease inhibitors can be specifically designed or selected to decrease the proteolysis of the zonulin and/or ZOT receptor agonist and/or the therapeutic agent.

### EXAMPLES

[³H]-Cyclosporin A (CsA; 8 Ci/mM, 1 mCi/ml) was purchased from Amersham Radiochemicals (Piscataway, N.J.). Ketamine HCl injection, USP, was purchased from Bedford Laboratories (Bedford, Ohio). [¹⁴C]-Mannitol (46.6 mCi/mM, 60 μCi/ml), benzalkonium chloride(BC), Xylazine, captopril, protease inhibitors (PI; bestatin and E-64) were purchased from Sigma Chemical Co. (St. Louis, Mo.). All chemicals were of analytical grade. All surgical supplies were purchased from World Precision Instruments (Sarasota, Fla.). Polyethylene 50 (PE50) tubing was obtained from Clay Adams (Parsippany, N.J.). Universol Scintillation counting cocktail was purchased from ICN (Cost Mesa, Calif.). The Caco-2 cell line was obtained from American Tissue Culture Collection (ATCC; Rockville, Md.). Caco-2 cell culture supplies (Dulbecco's modified Eagle medium, phosphate buffer saline (PBS), non essential amino acids, fetal bovine serum, L-glutamate, trypsin (0.25%)-EDTA (1 mM), and Penicillin G-streptomycin sulfate antibiotic mixture) were purchased from Gibco Laboratories (Lenexa, Kans.). Transwell clusters, 12-well (3 μm pores, surface area 1 cm²) were purchased from Corning Costar (Cambridge, Mass.).

Caco-2 cells, a human colon adenocarcinoma cell line, were grown as monolayers for 21 days in Dulbecco's Modified Eagle's medium (1×) containing 10% fetal bovine serum, 1% non-essential amino acid solution, 1% penicillin-streptomycin and 2% glutamine at 37° C. in an atmosphere of 5% CO₂ and 90% relative humidity. Caco-2 cells from passage numbers of 51 to 52 were seeded on permeable polycarbonate inserts (1 cm², 0.4 μm pore size) in 12 Transwell plates at a density of 80,000 cells/cm². The inserts were fed with media every other day until they were used for experiments 21 days after the initial seeding. The integrity of the cell monolayers was evaluated by measuring the transepithelial electrical resistance (TEER) values before the study using a Millicell®-ERS meter (Millipore Corp., Bedford, Mass.) with chopstick electrodes. The transport of [¹⁴C]-Mannitol was also performed prior to the transport studies. The cell monolayers were considered to be tight when the apparent permeability coefficients (Pₐₚₚ) value of [¹⁴C]-Mannitol was <1×10⁻⁶ cm/s. The cell monolayers were washed twice with PBS prior to the transport experiments. After the wash, the plates were incubated for 30 min at 37° C., and the integrity of the cell monolayers was evaluated by measurement of TEER. The cell inserts were used in transport experiments when the TEER values reached >300 Ωcm².

To measure the apical to basolateral transport of CsA, 0.5 ml of each CsA treatment, i.e., (1) the PBS solution of CsA, (2) the PBS solution of CsA/PI, (3) the PBS solution of CsA/PI/BC, (4) the PBS solution of CsA/AT1002, (5) the PBS solution of CsA/PI/AT1002, and (6) the PBS solution of CsA/PI/BC/AT1002 (CsA 0.5 μCi/ml, PI (bestatin 15 mM and E64 5 mM), BC 0.005 w/v %, and AT1002 5 mM, respectively) was added to the apical side, and 1.5 ml of PBS was added to the basolateral side of the insert. The insert was moved to a well containing fresh PBS every 10 min for 40 min. Samples were collected from the basolateral side of each well, and the radioactivity of CsA transported was measured by Beckman Coulter LS 6500 multi-purpose Scintillation counter.

Male Sprague-Dawley rats (230-280 g) were purchased from Harlan Laboratories (Indianapolis, Ind.). Rats were housed individually in cages and allowed to acclimate at least two days after arrival. Rats were fed Rat Chow and water ad libitum and maintained on a 12-h light: 12-h dark cycle. The protocol for the animal studies was approved by the School of Pharmacy, University of Maryland IACUC.

Peptides like AT1002, when administered orally, are likely to undergo substantial degradation in the stomach and gastrointestinal tract. In order to exclude the variability in effect as a result of gastric degradation, AT1002 was administered intraduodenally to rats, and plasma concentrations of CsA were monitored for 120 min. Male Sprague-Dawley rats were fasted overnight prior to and during the study with free access to water. Prior to the administration of AT1002, the rats were anesthetized with an intra-peritoneal injection of ketamine (80 mg/kg) and xylazine (12 mg/kg), and the duodenum and jugular vein were cannulated. Four treatments, i.e., (1) a distilled water solution of CsA (120 μCi/kg), (2) a distilled water solution of CsA/PI/BC (120 μCi/kg, PI (bestatin 30 mg/kg and E-64 10 mg/kg), BC 0.1% w/v, respectively), (3) a distilled water solution of CsA/AT1002 (120 μCi/kg and 40 mg/kg, respectively), and (4) distilled water solutions of the CsA/PI/BC/AT1002 (120 μCi/kg, PI (bestatin 30 mg/kg and E-64 10 mg/kg), BC 0.1% w/v, AT1002 doses of 5, 10 or 40 mg/kg in each group of study, respectively), were then slowly administered to intraduodenally cannulated rats with a volume dose of 2 ml/kg rat. Blood samples (250 μl) were drawn via the jugular cannula into heparinized syringes at 0 (actual time point was −5 min before the administration), 20, 60, and 120 min into polypropylene tubes, centrifuged (13,000 rpm for 10 min) immediately and plasma was obtained. Scintillation cocktail was added and samples were analyzed for radioactivity by Beckman Coulter LS 6500 multi-purpose Scintillation counter.

Pₐₚₚ was calculated according to the following equation:

$${P}_{app} = \frac{ⅆ Q}{ⅆ t} \frac{Vr}{A \cdot {D}_{0}}$$

Where dQ/dt is equal to the linear appearance rate of mass in the receiver solution, A is the cross sectional area (1 cm²), Dₒ is equal to the initial amount in the donor compartment, Vr is equal to the volume of the receiver compartment (1.5 ml).

The percent enhancement ratio, ER (%), for the Pₐₚₚ was calculated from the formula,

$$ER ( \% ) = \frac{{P}_{app \cdot ( treatment )}}{{P}_{app \cdot ( control )}} \times 100$$

In the in vivo study, the amount of radiolabelled CsA absorbed was converted to concentrations using the specific activity of the radiolabelled stock solution. The area under the plasma concentration-time curve (AUC₀₋ₜ) was calculated using the linear trapezoidal rule. The highest observable concentration was defined as maximum concentration (Cₘₐₓ). The percent enhancement ratio, ER (%), for the pharmacokinetic parameters was calculated from the formula,

$$ER ( \% ) = \frac{{PK}_{parameter \cdot ( treatment )}}{{PK}_{parameter \cdot ( control )}} \times 100$$

All data were expressed as the mean and standard error of the mean of the values (mean±SEM). The statistical significance of differences between treatments and/or controls was evaluated using the Student's t-test and Analysis of variance followed by Dunnett's post hoc test (SPSS for Windows versions 12.0., SPSS Inc., Chicago, Ill.) (p<0.05 or p<0.01).

### Example 1

Caco-2 transport studies of CsA with AT1002

Table 1 summarizes the permeability coefficients (Pₐₚₚ) associated with the various transport studies performed with AT1002 and CsA. The apparent permeability coefficient (Pₐₚₚ) of Mannitol, CsA, and CsA treatments across Caco-2 cell monolayers. (Mannitol 0.5 μCi/ml, CsA 0.5 μCi/ml, PI (bestatin 15 mM and E-64 5 mM), BC 0.005 w/v %, and/or AT1002 5 mM, respectively). Data presented as mean±SEM (n=3).

TABLE I

| | Pₐₚₚ (×10⁻⁶ cm/sec) | ER (%) |
|---|---|---|
| Mannitol | 0.69 ± 0.06 | — |
| CsA | 1.28 ± 0.10 | — |
| CsA/AT1002 | 1.54 ± 0.13 | 120 |
| CsA/PI | 1.59 ± 0.07 | — |
| CsA/PI/AT1002 | 1.76 ± 0.05 | 111 |
| CsA/PI/BC | 1.60 ± 0.03 | — |
| CsA/PI/BC/AT1002 | 1.52 ± 0.06 | 95 |

The mean Pₐₚₚ determined for CsA were 1.28±0.10, 1.54±0.13, 1.59±0.07, 1.76±0.05, 1.60±0.03, and 1.52±0.06 (×10⁻⁶ cm/sec, mean±SEM, n=3), for the following treatments CsA, CsA/AT1002, CsA/PI, CsA/PI/AT1002, CsA/PI/BC, and CsA/PI/BC/AT1002, respectively. The fold increases of CsA across Caco-2 cell monolayers were 120%, 111%, and 95% after the following treatments CsA/AT1002, CsA/PI/AT1002, and CsA/PI/BC/AT1002 treatment compared to each of the following controls, CsA, CsA/PI, and CsA/PI/BC, respectively. However, there were no significant differences observed in Papp or fold-increase for the transport of CsA across Caco-2 cell monolayers between treatments and each control. Mannitol permeability was found to be 6.86±0.57×10⁻⁷ cm/sec suggesting integrity of the tight junctions in the Caco-2 cells.

### Example 2

Intra-duodenal administration of CsA with AT1002 to rats

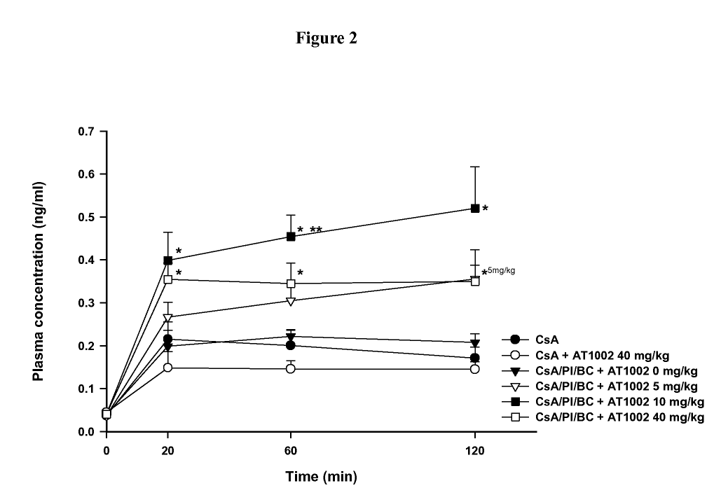

FIG. 2 illustrates the mean (±SEM) plasma concentration versus time profile for CsA in jugular vein cannulated Sprague-Dawley rats following the ID administration of four treatments of CsA, i.e., CsA, CsA/PI/BC, CsA/AT1002, and CsA/PI/BC/AT1002 (at AT1002 doses of 5, 10 or 40 mg/kg). The plasma concentration of CsA from CsA/PI/BC/AT1002 with the dose of 40 mg of AT1002 were 178% and 155% significantly (p<0.05) higher than those from CsA/PI/BC as the control at 20 min and 60 min time period respectively. Further, the plasma concentration of CsA was significantly increased by 201% (p<0.05), 205% (p<0.01), and 250% (p<0.05) from the dose of 10 mg/kg of AT1002 compared to the control at each 20 min, 60 min, and 120 min time period, respectively. Also, the plasma concentration of CsA from the dose of 5 mg of AT1002 were 134% significantly (p<0.05) higher than the control at 20 min time period, indicating a significant enhancement in absorption of CsA by AT1002. In comparison, no significant differences were found in the plasma concentration of CsA between the CsA, CsA/PI/BC, and CsA/AT1002 solutions at time points evaluated.

The AT1002 treatments (CsA/PI/BC with AT1002 10 mg/kg or 40 mg/kg) were found to significantly (p<0.01) to increase the extent (AUC_{0-120 min}; 50.70±1.78 min ng/ml, 214%, and 38.81±4.27 min ng/ml, 164%, respectively) and rate (Cₘₐₓ; 0.62±0.03 ng/ml, 256%, and 0.43±0.06 ng/ml, 177%, respectively) as compared to extent (AUC_{0-120 min}; 23.70±1.79 min ng/ml) and rate (Cₘₐₓ; 0.24±0.02 ng/ml) observed with the control treatment (CsA/PI/BC). On the contrary, CsA/PI/AT1002 5 mg/kg led to a 145% increase in the AUC_{0-120 min} (34.28±3.23 min ng/ml) and 146% (0.36±0.03 ng/ml) increase in Cₘₐₓ with non-significant differences as compared the control treatment (CsA/PI/BC), and CsA/AT1002 40 mg/kg without PI/BC displayed a non-significant decreased in AUC_{0-120 min} and Cₘₐₓ as compared CsA treatment. Further, the increase in AUC_{0-120 min} and Cₘₐₓ was not statistically different for the CsA/PI/BC without AT1002 compared with those of CsA. (Table II). Table II shows the results.

Mean±SEM bioavailability parameters for CsA (120 μCi/kg) after ID administration to jugular vein cannulated Sprague-Dawley rats (n=4-5) alone and/or with PI/BC (PI(bestatin 30 mg/kg and E-64 10 mg/kg) and BC 0.1 w/v %) and/or AT1002. *Significant (p<0.01) compared to CsA and CsA/PI/BC.

TABLE II

| | AUC_{0-120 min} | ER | Cₘₐₓ | ER |
|---|---|---|---|---|
| | (min ng/ml) | (%) | (ng/ml) | (%) |
| CsA | 21.97 ± 3.79 | — | 0.22 ± 0.04 | — |
| CsA+ AT1002 | 16.56 ± 1.81 | 75 | 0.18 ± 0.03 | 83 |
| 40 mg/kg | | | | |
| CsA/PI/BC+ AT1002 | 23.70 ± 1.79 | — | 0.24 ± 0.02 | — |
| 0 mg/kg | | | | |
| CsA/PI/BC+ AT1002 | 34.28 ± 3.23 | 145 | 0.36 ± 0.03 | 146 |
| 5 mg/kg | | | | |
| CsA/PI/BC+ AT1002 | 50.70 ± 1.78 * | 214 | 0.62 ± 0.03 * | 256 |
| 10 mg/kg | | | | |
| CsA/PI/BC+ AT1002 | 38.81 ± 4.27 * | 164 | 0.43 ± 0.06 * | 177 |
| 40 mg/kg | | | | |

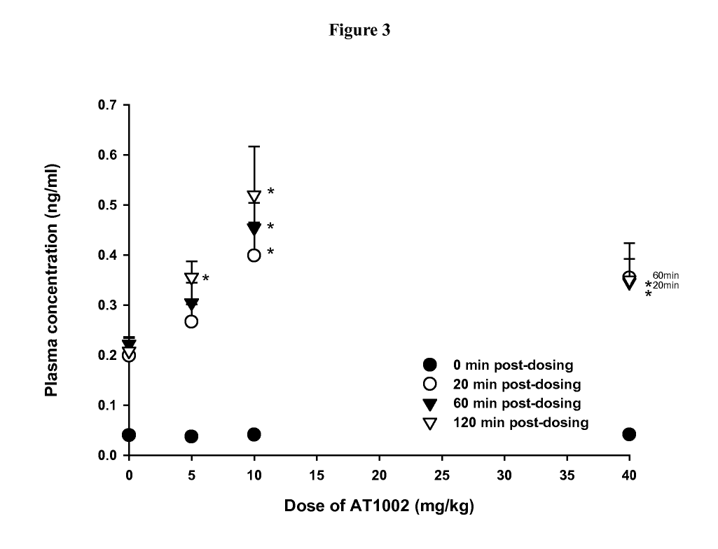

The influence of increasing doses of AT1002 (0, 5, 10, and 40 mg/kg) on the plasma concentration of CsA are shown in FIG. 3. The plasma concentrations of CsA at each sampling time point correlated well with the dose range over 0 mg/kg to 10 mg/kg of AT1002, with r² of 0.9665 at 20 min, 0.9731 at 60 min, and 0.9991 at 120 min. No statistically significant change in the plasma concentration of CsA between 10 mg/kg and 40 mg/kg of AT1002 at each time point, suggesting that the increase of CsA reached a stead state from 10 mg/kg of AT1002.

Many therapeutically active agents experience low bioavailability after oral administration due to poor absorption or susceptibility to first pass metabolism. Transient opening of TJ to improve paracellular drug transport and increase oral absorption would be beneficial to the therapeutic effect. Absorption enhancers are capable of modulation of TJ to improve the transport or absorption of low bioavailable drugs. However, some absorption enhancers cause serious damage to the epithelial integrity, morphology and function (13). Our studies examined the effect of AT1002 as an absorption enhancer of CsA, one of the major potent immunosuppressive drugs which exhibits a low therapeutic index and a poor BA with a mean of ˜20% (12), on Caco-2 cell monolayers and after intraduodenal administration in male Sprague-Dawley rats.

As previously noted, one of the disadvantages of performing in vivo studies with ZOT or ΔG is that the isolation and purification protocol is tedious and time consuming and more importantly the yield of protein is not sufficient to conduct in vivo studies. For this reason, investigations were performed to identify a fragment of ΔG, an amino acid sequence that presumably retained the permeating effects of ΔG but would be amenable to synthesis. Studies were performed by Fasano et al to identify this fragment, referred to as AT1002 (8). As stated, AT1002 was synthesized as assumed to retain the ZOT and/or ΔG permeating effect on intercellular TJ. ZOT, a toxin produced by the bacterial strain V. cholerae, binds to a specific receptor on the luminal surface of the intestine and reversibly opening the TJ between intestinal epithelial cells (2-7). ΔG, a biologically active 12 kDa fragment of ZOT, was isolated and displayed the intrinsic activity of reversibly modulating TJ thus increasing the paracellular transport of drugs (8). ZOT and ΔG triggers a cascade of intracellular events mediated by protein kinase C with polymerization of soluble G-actin, subsequent displacement of proteins from the junctional complex, and loosening of TJ (3). Thus, they can reversibly open the intestinal TJ in a non toxic manner (2-7,10).

In previous studies with ZOT, bioavailability of oral insulin coadministered with ZOT (4.4×10⁻¹⁰ mol/kg) was sufficient to lower serum glucose concentrations to levels comparable to those obtained after parenteral injection of the hormone in diabetic rats (4). ZOT (0.45×10⁻¹⁰ mol/ml, 0.89×10⁻¹⁰ mol/ml) increased the permeability of molecular weight markers (sucrose, Inulin) over a range of 130% to 195% and chemotherapeutic agents (paclitaxel, doxorubicin) across the bovine brain microvessel endothelial cells (BBMEC) (14). And, ZOT (0.22 to 0.89×10⁻¹⁰ mol/ml) enhanced the transport of varying molecular weights (mannitol, PEG4000, Inulin) or low bioavailability (doxorubicin, paclitaxel, acyclovir, cyclosporin A, acticonvulsant enaminones) up to 30 fold as seen with paclitaxel across Caco-2 cell monolayers, without modulating the transcellular transport (6,7).

Also, studies have shown that ΔG (0.83 to 1.50×10⁻⁸ mol/ml) increased the transport of paracellular markers (mannitol, Inulin, PEG4000) by 1.2 to 2.8-fold across Caco-2 cells relative to the transepithelial transport of markers in its absence (9,10), and after ID administration to rats, ΔG (3.48 to 6.00×10⁻⁸ mol/kg) displayed high intrinsic biological activity with paracellular markers (mannitol, Inulin, PEG4000) and some low bioavailable drugs (CsA, ritonavir, saquinavir, acyclovir) (9-11). Moreover, the in vivo studies with ΔG displayed up to 57 and 50-fold increased in Cₘₐₓ and AUC as seen with CsA after metabolic protection was provided (11).

A significant enhancement in the absorption of CsA was observed in this study after dosing with AT1002, suggesting that the six-mer peptide retained the ZOT domain directly involved in the protein permeating effect. AT1002 statistically and significantly increased AUC_{0-120 min} of CsA over a range of 164% to 214%, and Cₘₐₓ of CsA over a range of 177% to 256% at 10 mg/kg (1.41×10⁻⁵ mol/kg) and 40 mg/kg (5.65×10⁻⁵ mol/kg) dose of AT1002 (p<0.01) from the treatment of CsA/PI/BC/AT1002 compared to CsA/PI/BC as control. Also, the plasma concentration of CsA was statistically and significantly increased over a range of 201% to 250% from CsA/PI/BC/AT1002 10 mg/kg compared to the CsA concentration of CsA/PI/BC at every time period examined in rats.

It was reported that protease inhibitors (a mixture of bestatin, captopril, and leupeptin) are needed to minimize enzymatic degradation of ΔG secondary to proteases or peptidases and to display a high intrinsic biological activity of drug with ΔG (9,11). Similarly, based on the low molecular weight and the peptide nature of AT1002, it would be expected that AT1002 would be extensively metabolized in the gastrointestinal track by enzymes and intestinal flora. When CsA was coadministered intraduodenally to rats with AT1002 excluding PI, the plasma concentration at each time period and bioavailability parameters (AUC_{0-120 min}, Cₘₐₓ) were not significantly changed compared to those of CsA. PI which was composed of bestatin and E-64 was selected in one of the treatment arms to minimize enzymatic degradation secondary to proteases or peptidases because of their inhibitory effect on leucine aminopeptidase, alanyl aminopeptidase, serine and cysteine proteases. In addition, previous studies have evaluated the use of BC in stabilizing peptide (15). Systematic investigations are underway in our lab to optimize the use of BC and AT1002 by LC-MS. Studies of the effect of PI/BC showed that PI/BC caused no significant difference in the absorption of CsA. Therefore, administration of PI/BC did not result in significant absorption improvement, and PI/BC/AT1002 absorption enhancement is due to metabolic protection and/or stabilizing effect of AT1002.

Upon this transport study across Caco-2 cells, no treatments showed statistically increase in their transport in the presence of AT1002 across cell monolayers compared to each control. The Caco-2 cell monolayers have been reported to have lower paracellular permeability than the intestinal epithelium (16-17) due to anatomical differences between intestinal segments and by noting the colonic origin of this cell line (18), and there are differences in the level of expression and substrate specificity of transporters and enzymes (16,19-21).

The enhancement of CsA by AT1002 is assumed to be related to protease activated receptor-2 (PAR-2) receptor. PAR-2 agonists are 6-mer peptides, with 4 of the amino acids being identical to that of the ZOT/Zonulin receptor binding motif (XX—IGRL) (8). This suggest that AT1002 (H—FCIGRL-OH) may possess similar biological activity at PAR-2 receptors. The PAR-2 receptor belongs to a class of G-protein coupled receptors that are activated by cleavage of their N-terminal by a proteolytic enzyme. Following the cleavage the newly unmasked N-terminal acts as a tethered ligand and activates the receptor (22). Intracolonic infusion of a 5 μg dose of the PAR-2 agonist, SLIGRL, resulted in a 2-fold increase in the paracellular permeability of [⁵¹C]-EDTA (23). Thus, the difference in the in vitro versus in vivo extent of enhancement observed in our studies might arise from differences in the expression of PAR-2 receptors along the gastrointestinal tract.

This study provided information on the effectiveness of the active fragment of ZOT and/or ΔG, AT1002, in enhancing in vivo oral absorption. The enhancing effects observed in vivo (CsA/PI/BC/AT1002) were found to be significantly higher than our controls (CsA or CsA/PI/BC), however, its effect in the in vitro model was not apparent. The in vivo intraduodenal absorption study illustrates AT1002's potential usefulness in enhancing oral drug delivery. Formulations of the peptide to minimize the adverse effects related to the physiology of the GI tract, will be useful and lead to the development of a practically relevant drug delivery technology for low bioavailable drugs.

### Example 2

Our recent observation that zonulin may represent a new member of the serine protease family whose target receptor seems to be a variant of the protease activated receptor (PAR)2, lead us to the observation that the first six amino acids following V. cholerae-mediated ZOT cleavage (AA 289-295 [FCIGRL]) closely resembles the active motif of PAR2 (SLIGRL). Therefore, the six-mer synthetic peptide FCIGRL (that we named AT1002) was generated. When tested in the Ussing chamber model, AT1002 retained the ZOT permeating effect on intercellular tight junctions.

We looked at the oral and intraduodenal dosing, the dose in mice, and rats as wintra-arterial and intravenouse dosing of AT1002 as well as doses to determine route and dose level.

The intestinal membrane transport study of [14C]-mannitol on the co-administration with AT-1002 in mice.

The ability of AT-1002 on the transport of [¹⁴C]-mannitol across the intestinal membrane was examined in mice. [¹⁴C]-mannitol (30 μCi/kg) was co-administered with AT-1002 (10 mg/kg) and protease inhibitors (i.e. bestatine (30 mg/kg) and E-64 (10 mg/kg)) to mice (n=3 per group) via the oral administration. The intestinal membrane transport of [³H]-sucrose was not enhanced at each sampling time.

The intestinal transport study of [14C]-mannitol on the co-administration with AT-1002 in rats.

The ability of AT-1002 on the transport of [¹⁴C]-mannitol across the intestine was examined in rats. [¹⁴C]-mannitol (30 μCi/kg) was co-administered with AT-1002 (10, 20 and 30 mg/kg), protease inhibitors (i.e. bestatine (30 mg/kg) and E-64 (10 mg/kg)) and benzalkonium chloride (0.05% w/v) to rats (n=3 per group) via the intra-duodenal administration. The intestinal membrane transport of [¹⁴C]-mannitol was not statistically enhanced but increased to the 1.43, 1.24 and 1.81 fold for the each dose of AT-1002, respectively by AT-1002.

The intestinal membrane transport study of [14C]-innulin on the co-administration with AT-1002 in mice.

The effect of AT-1002 on the transport of [¹⁴C]-innulin across the intestine was examined in mice. [¹⁴C]-innulin (30 μCi/kg) was co-administered with AT-1002 (10, 20 and 40 mg/kg), protease inhibitors (i.e. bestatine (30 mg/kg) and E-64 (10 mg/kg)) and benzalkonium chloride (0.05% w/v) to rats (n=4-5 per group) via the intra-duodenal administration. The intestinal membrane transport of [¹⁴C]-innulin was not enhanced at each dose of AT-1002.

The intestinal transport study of [³H]-cyclosporin A on the co-administration with AT-1002 in rats.

The ability of AT-1002 on the transport of [³H]-cyclosporin across the intestine was examined in rats. [³H]-cyclosporin (120 μCi/kg) was co-administered with AT-1002 (40 mg/kg), protease inhibitors (i.e. bestatine (30 mg/kg) and E-64 (10 mg/kg)) and benzalkonium chloride (0.1% w/v) to rats (n=5 per group) via the intra-duodenal administration. The intestinal transport of [³H]-cyclosporin A was statistically enhanced to the 1.78 and 1.55 fold for the 20 min and 60 min of the sampling time, respectively by AT-1002.

B. J. Aungst. Intestinal Permeation enhancers. J Pharm Sci. 89(4):429-442 (2000).

A. Fasano, B. Baudry D. W. Pumplin, S. S. Wasserman, B. D. Tall, J. M. Ketley, and J. B. Kaper. Vibrio cholerae produces a second enterotoxin, which affects intestinal tight junctions. Proc Natl Acad Sci USA. 88(12):5242-5246 (1991).

A. Fasano, C. Fiorentini, G. Donelli, S. Uzzau, J. B. Kaper, K. Margaretten, X. Ding, S. Guandalini, L. Comstock, and S. E. Goldblum. Zonula occludens toxin modulates tight junctions through protein kinase C-dependent actin reorganization, in vitro. J Clin Invest. 96(2):710-720 (1995).

A. Fasano and S. Uzzau. Modulation of intestinal tight junctions by Zonula occludens toxin permits enteral administration of insulin and other macromolecules in an animal model. J Clin Invest. 99(6):1158-1164 (1997).

A. Fasano, S. Uzzau, C. Fiore, and K. Margaretten. The enterotoxic effect of zonula occludens toxin on rabbit small intestine involves the paracellular pathway. Gastroenterology. 112(3):839-846 (1997).

D. S. Cox, H. Gao, S. Raje, K. R. Scott, and N. D. Eddington. Enhancing the permeation of marker compounds and enaminone anticonvulsants across Caco-2 monolayers by modulating tight junctions using zonula occludens toxin. Eur J Pharm Biopharm. 52(2):145-150 (2001).

D. S. Cox, S. Raje, H. Gao, N. N. Salama, and N. D. Eddington. Enhanced permeability of molecular weight markers and poorly bioavailable compounds across Caco-2 cell monolayers using the absorption enhancer, zonula occludens toxin. Pharm Res. 19(11):1680-1688 (2002).

M. Di Pierro, R. Lu, S. Uzzau, W. Wang, K. Margaretten, C. Pazzani, F. Maimone, and A. Fasano. Zonula occludens toxin structure-function analysis. Identification of the fragment biologically active on tight junctions and of the zonulin receptor binding domain. J Biol Chem. 276(22):19160-19165 (2001).

N. N. Salama, A. Fasano, R. Lu, and N. D. Eddington. Effect of the biologically active fragment of zonula occludens toxin, delta G, on the intestinal paracellular transport and oral absorption of mannitol. Int J Pharm. 251(1-2):113-121 (2003).

N. N. Salama, A. Fasano, M. Thakar, and N. D. Eddington. The effect of delta G on the transport and oral absorption of macromolecules. J Pharm Sci. 93(5):1310-1319 (2004).

N. N. Salama, A. Fasano, M. Thakar, and N. D. Eddington. The impact of DeltaG on the oral bioavailability of low bioavailable therapeutic agents. J Pharmacol Exp Ther. 312(1):199-205 (2005).

Y. Ogino, E. Kobayashi, and A. Fujimura. Comparison of cyclosporin A and tacrolimus concentrations in whole blood between jejunal and ileal transplanted rats. J Pharm Pharmacol. 51(7):811-815 (1999).

M. Thanou, B. I. Florea, M. W. Langemeyer, J. C. Verhoef, H. E. Junginger. N-trimethylated chitosan chloride (TMC) improves the intestinal permeation of the peptide drug buserelin in vitro (Caco-2 cells) and in vivo (rats). Pharm Res. 17(1):27-31 (2000).

C. S. Karyekar, A. Fasano, S. Raje, R. Lu, T. C. Dowling, and N. D. Eddington N D. Zonula occludens toxin increases the permeability of molecular weight markers and chemotherapeutic agents across the bovine brain microvessel endothelial cells. J Pharm Sci. 92(2):414-423 (2003).

K. H. Song, H. M. An, H. J. Kim, S. H. Ahn, S. J. Chung, and C. K. Shim. Simple liquid chromatography-electrospray ionization mass spectrometry method for the routine determination of salmon calcitonin in serum. J Chromatogr B Analyt Technol Biomed Life Sci. 775(2):247-255 (2002).

P. Artursson, R. T. Borchardt. Intestinal drug absorption and metabolism in cell cultures: Caco-2 and beyond. Pharm Res. 14(12):1655-1658 (1997).

W. Rubas, M. E. Cromwell, Z. Shahrokh, J. Villagran, T. N. Nguyen, M. Wellton, T. H. Nguyen, and R. J. Mrsny. Flux measurements across Caco-2 monolayers may predict transport in human large intestinal tissue. J Pharm Sci. 85(2):165-169 (1996).

F. A. Wilson and J. M. Dietschy. Characterization of bile acid absorption across the unstirred water layer and brush border of the rat jejunum. J Clin Invest. 51(12):3015-3025 (1972).

K. L. Audus, R. L. Bartel, I. J. Hidalgo, R. T. Borchardt. The use of cultured epithelial and endothelial cells for drug transport and metabolism studies. Pharm Res. 7(5):435-451 (1990).

R. Borchardt. The application of cell culture systems in drug discovery and development. J Drug Target. 3(3):179-182 (1995).

D. Brayden. Human intestinal epithelial cell monolayers as pre-screens for oral drug delivery. Pharm News 4:11-15 (1997).

S. Nystedt, K. Emilsson, A. K. Larsson, B. Strombeck, and J. Sundelin. Molecular cloning and functional expression of the gene encoding the human proteinase-activated receptor 2. Eur J Biochem. 232(1):84-89 (1995).

N. Cenac, A. C. Chin, R. Garcia-Villar, C. Salvador-Cartier, L. Ferrier, N. Vergnolle, A. G. Buret, J. Fioramonti, and L. Bueno. PAR2 activation alters colonic paracellular permeability in mice via IFN-gamma-dependent and -independent pathways. J Physiol. 558(Pt 3):913-925 (2004).

What is claimed is:

1. A therapeutic composition comprising
a therapeutically effective amount of one or more therapeutic agents; and
an intestinal absorption enhancing amount of one or more tight junction agonists.

2. The composition of claim 1 wherein at least one of the tight junction agonists is a zonulin and/or ZOT receptor agonist.

3. The composition of claim 1, wherein at least one tight junction agonist comprises a peptide.

4. The composition of claim 3 wherein the peptide comprises from about 6 to about 15 amino acid residues.

5. The composition of claim 3 wherein the peptide comprises from about 6 to about 9 amino acid residues.

6. The composition of claim 3 wherein the peptide comprises a sequence selected from the group consisting of FCIGRX, FCIGXL, FCIXRL, FCXGRL, FXIGRL, XCIGRL, XXIGRL, XCXGRL, XCIXRL, XCIGXL, XCIGRX, FXXGRL, FXIXRL, FXIGXL, FXIGRX, FCXXRL, FCXGXL, FCXGRX, FCIXXL, FCIXRX, and FCIGXX, wherein each X is independently a natural or synthetic amino acid residue.

7. The composition of claim 2 wherein at least one of the one or more zonulin and/or ZOT receptors is a peptide comprising the sequence FCIGRL.

8. The composition of claim 1 wherein at least one therapeutic agent is selected from the group consisting of an antibiotic, an anti-inflammatory, an analgesic, an immunosuppressant, and a peptide hormone.

9. The composition of claim 8 wherein the immunosuppressant is selected from the group consisting of cyclosporin A, FK506, prednisone, methylprednisolone, cyclophosphamide, thalidomide, azathioprine, and daclizumab, physalin B, physalin F, physalin G, seco-steroids purified from Physalis angulata L., DSG(15-deoxyspergualin, 15-dos), MMF, rapamycin and its derivatives, CCI-779, FR 900520, FR 900523, NK86-1086, depsidomycin, kanglemycin-C, spergualin, prodigiosin25-c, cammunomicin, demethomycin, tetranactln, tranilast, stevastelins, myriocin, gllooxin, FR 651814, SDZ214-104, bredinin, WS9482, mycophenolic acid, 15-deoxyspergualin, mimoribine, misoprostol, OKT3, anti-IL-2 receptor antibodies, azasporine, leflunomide, mizoribine, azaspirane (SKF 105685), paclitaxel, altretamine, busulfan, chlorambucil, ifosfamide, mechlorethamine, melphalan, thiotepa, cladribine, fluorouracil, floxuridine, gemcitabine, thioguanine, pentostatin, methotrexate, 6-mercaptopurine, cytarabine, carmustine, lomustine, streptozotocin, carboplatin, cisplatin, oxaliplatin, iproplatin, tetraplatin, lobaplatin, JM216, JM335, fludarabine, aminoglutethimide, flutamide, goserelin, leuprolide, megestrol acetate, cyproterone acetate, tamoxifen, anastrozole, bicalutamide, dexamethasone, diethylstilbestrol, bleomycin, dactinomycin, daunorubicin, doxirubicin, idarubicin, mitoxantrone, losoxantrone, mitomycin-c, plicamycin, paclitaxel, docetaxel, topotecan, irinotecan, 9-amino camptothecan, 9-nitro camptothecan, GS-211, etoposide, teniposide, vinblastine, vincristine, vinorelbine, procarbazine, asparaginase, pegaspargase, octreotide, estramustine, and hydroxyurea, and combinations thereof.

10. The composition of claim 9 wherein the immunosuppressant is cyclosporin A.

11. The composition of claim 8 wherein the peptide hormone is insulin.

12. The composition of claim 1 wherein at least one of the one or more therapeutic agents is selected from the group consisting of a small molecule, a peptide, a protein, a lipid, a carbohydrate, and combinations thereof.

13. The composition of claim 1 wherein at least one of the one or more therapeutic agents is selected from the group consisting of a chemotherapeutic, a gene therapy vector, a growth factor, parathyroid hormone, human growth hormone, a contrast agent, an angiogenesis factor, a radionuclide, an anti-infection agent, an anti-tumor compound, a receptor-bound agent, a hormone, a steroid, a protein, a complexing agent, a polymer, heparin, covalent heparin, ar thrombin inhibitor, hirudin, hirulog, argatroban, D-phenylalanyl-L-poly-L-arginyl chloromethyl ketone, an antithrombogenic agent, urokinase, streptokinase, a tissue plasminogen activator, a thrombolytic agent, a fibrinolytic agent, a vasospasm inhibitor, a calcium channel blocker, a nitrate, nitric oxide, a nitric oxide promoter, a vasodilator, an antihypertensive agent, an antimicrobial agent, an antibiotic, aspirin, triclopidine, a glycoprotein IIb/IIIa inhibitor, an inhibitor of surface glycoprotein receptors, an antiplatelet agent, colchicine, an antimitotic, a microtubule inhibitor, dimethyl sulfoxide (DMSO), a retinoid, an antisecretory agent, cytochalasin, an actin inhibitor, a remodeling inhibitor, deoxyribonucleic acid, an antisense nucleotide, an agent for molecular genetic intervention, methotrexate, an antimetabolite, an antiproliferative agent, tamoxifen citrate, an anti-cancer agent, dexamethasone, dexamethasone sodium phosphate, dexamethasone acetate,a dexamethasone derivative, an anti-inflammatory steroid, a non-steroidal antiinflammatory agent, cyclosporin, an immunosuppressive agent, trapidal, a PDGF antagonist, angiopeptin, a growth hormone antagonist, angiogenin, a growth factor antibody, an anti-growth factor antibody, a growth factor antagonist, dopamine, bromocriptine mesylate, pergolide mesylate, a dopamine agonist, ⁶⁰Co, ¹⁹²Ir, ³²P, ¹¹¹In, ⁹⁰Y, ⁹⁹mTc, a radiotherapeutic agent, an iodine-containing compound, a barium-containing compound, gold, tantalum, platinum, tungsten, a heavy metal functioning as a radiopaque agent, a peptide, a protein, an enzyme, an extracellular matrix component, a cellular component, captopril, enalapril, an angiotensin converting enzyme (ACE) inhibitor, ascorbic acid, α-tocopherol, superoxide dismutase, deferoxamine, a 21-aminosteroid (lasaroid), a free radical scavenger, an iron chelator, an antioxidant, a ¹⁴C—, ³H—, ¹³¹I—, ³²P— or ³⁶S-radiolabelled form or other radiolabelled form of any of the foregoing, estrogen, a sex hormone, AZT, an antipolymerases, acyclovir, famciclovir, rimantadine hydrochloride, ganciclovir sodium, an antiviral agents, 5-aminolevulinic acid, meta-tetrahydroxyphenylchlorin, hexadecafluoro zinc phthalocyanine, tetramethyl hematoporphyrin, rhodamine 123 or other photodynamic therapy agents, an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents, gene therapy agents, enalapril, a prodrug, and an agent for treating benign prostatic hyperplasia (BHP), or combinations thereof.

14. The composition of claim 1 wherein the composition is in aqueous solution.

15. The composition of claim 1 further comprising one or more protease inhibitors.

16. The composition of claim 15 wherein at least one of the one or more protease inhibitors is selected from the group consisting of bestatin, L-trans-3-carboxyoxiran-2-carbonyl-L-leucylagmatine, EDTA, PMSF, aprotinin, amyloid protein precursor (APP), amyloid beta precursor protein, α₁-proteinase inhibitor, collagen VI, and bovine pancreatic trypsin inhibitor (BPTI), 4-(2-aminoethyl)-benzenesulfonyl fluoride (AEBSF), antipain, benzamidine, chymostatin, ε-aminocaproate, N-ethylmaleimide, leupeptin, pepstatin A, phosphoramidon, and combinations thereof.

17. The composition of claim 1 further comprising one or more pharmaceutically acceptable excipients.

18. The composition of claim 17 wherein at least one of the tight junction agonists is one or more peptide zonulin and/or ZOT receptors agonists comprising the sequence FCIGRL and the composition further comprises at least one protease inhibitor and one or more therapeutic agents selected from the group consisting of a small molecule, a peptide, a protein, a lipid, and a carbohydrate, and combinations thereof.

19. A method of treating a subject comprising orally administering to the subject a composition comprising one or more therapeutic agents and an intestinal absorption enhancing amount of one or more tight junction agonists.

20. The method according to claim 19, wherein at least one tight junction agonist is a zonulin and/or ZOT receptor agonist.

21. The method of claim 19 wherein the subject is a mammal.

22. The method of claim 19 wherein the subject is a human.

23. The method of claim 20 wherein at least one of the one or more zonulin and/or ZOT receptors agonists comprises a peptide.

24. The method of claim 23 wherein the peptide comprises from about 6 to about 15 amino acid residues.

25. The method of claim 23 wherein the peptide comprises from about 6 to about 9 amino acid residues.

26. The method of claim 23 wherein the peptide comprises a sequence selected from the group consisting of FCIGRX, FCIGXL, FCIXRL, FCXGRL, FXIGRL, XCIGRL, XXIGRL, XCXGRL, XCIXRL, XCIGXL, XCIGRX, FXXGRL, FXIXRL, FXIGXL, FXIGRX, FCXXRL, FCXGXL, FCXGRX, FCIXXL, FCIXRX, and FCIGXX, wherein X is independently a natural or synthetic amino acid residue.

27. The method of claim 23 wherein at least one of the one or more zonulin and/or ZOT receptors agonists is a peptide comprising the sequence FCIGRL.

28. The method of claim 19 wherein at least one of the one or more therapeutic agents is selected from the group consisting of an antibiotic, an anti-inflammatory, an analgesic, an immunosuppressant, and a peptide hormone.

29. The method of claim 28 wherein the immunosuppressant is selected from the group consisting of cyclosporin A, FK506, prednisone, methylprednisolone, cyclophosphamide, thalidomide, azathioprine, and daclizumab, physalin B, physalin F, physalin G, seco-steroids purified from Physalis angulata L., DSG(15-deoxyspergualin, 15-dos), MMF, rapamycin and its derivatives, CCI-779, FR 900520, FR 900523, NK86-1086, depsidomycin, kanglemycin-C, spergualin, prodigiosin25-c, cammunomicin, demethomycin, tetranactln, tranilast, stevastelins, myriocin, gllooxin, FR 651814, SDZ214-104, bredinin, WS9482, mycophenolic acid, 15-deoxyspergualin, mimoribine, misoprostol, OKT3, anti-IL-2 receptor antibodies, azasporine, leflunomide, mizoribine, azaspirane (SKF 105685)), paclitaxel, altretamine, busulfan, chlorambucil, ifosfamide, mechlorethamine, melphalan, thiotepa, cladribine, fluorouracil, floxuridine, gemcitabine, thioguanine, pentostatin, methotrexate, 6-mercaptopurine, cytarabine, carmustine, lomustine, streptozotocin, carboplatin, cisplatin, oxaliplatin, iproplatin, tetraplatin, lobaplatin, JM216, JM335, fludarabine, aminoglutethimide, flutamide, goserelin, leuprolide, megestrol acetate, cyproterone acetate, tamoxifen, anastrozole, bicalutamide, dexamethasone, diethylstilbestrol, bleomycin, dactinomycin, daunorubicin, doxirubicin, idarubicin, mitoxantrone, losoxantrone, mitomycin-c, plicamycin, paclitaxel, docetaxel, topotecan, irinotecan, 9-amino camptothecan, 9-nitro camptothecan, GS-211, etoposide, teniposide, vinblastine, vincristine, vinorelbine, procarbazine, asparaginase, pegaspargase, octreotide, estramustine, and hydroxyurea, and combinations thereof.

30. The method of claim 28 wherein the immunosuppressant is cyclosporin A.

31. The method of claim 28 wherein the peptide hormone is insulin.

32. The method of claim 19 wherein at least one of the one or more therapeutic agents is selected from the group consisting of a small molecule, a peptide, a protein, a lipid, a carbohydrate, and combinations thereof.

33. The method of claim 19 wherein the composition is in aqueous solution.

34. The method of claim 19 further comprising one or more protease inhibitors.

35. The method of claim 33 wherein at least one of the one or more protease inhibitors is selected from the group consisting of bestatin, L-trans-3-carboxyoxiran-2-carbonyl-L-leucylagmatine, EDTA, PMSF, aprotinin, amyloid protein precursor (APP), amyloid beta precursor protein, α₁-proteinase inhibitor, collagen VI, and bovine pancreatic trypsin inhibitor (BPTI), 4-(2-aminoethyl)-benzenesulfonyl fluoride (AEBSF), antipain, benzamidine, chymostatin, ε-aminocaproate, N-ethylmaleimide, leupeptin, pepstatin A, phosphoramidon, and combinations thereof.

36. The method of claim 19 wherein the composition further comprises one or more pharmaceutically acceptable excipients.

37. The method of claim 36 wherein at least one of the tight junction agonists comprises one or more peptide zonulin and/or ZOT receptors agonists comprising the sequence FCIGRL and the composition further comprises at least one protease inhibitor and one or more therapeutic agents selected from the group consisting of a small molecule, a peptide, a protein, a lipid, and a carbohydrate, and combinations thereof.

38. The method of claim 19 wherein orally administering comprises administering to the gut.

39. A method of treating diabetes in an animal in need thereof, comprising:
orally administering to the animal a composition comprising insulin, a derivative of insulin, or a combination thereof, and an intestinal absorption enhancing amount of one or more tight junction agonists.

40. The method of claim 39, wherein at least one tight junction agonist comprises at least one zonulin and/or ZOT receptor agonist.

41. The method of claim 39 wherein the animal is a mammal.

42. The method of claim 39 wherein the animal is a human.

43. The method of claim 40 wherein at least one of the one or more zonulin and/or ZOT receptors agonists comprises a peptide.

44. The method of claim 43 wherein the peptide comprises from about 6 to about 15 amino acid residues.

45. The method of claim 43 wherein the peptide comprises from about 6 to about 9 amino acid residues.

46. The method of claim 43 wherein the peptide is selected from the group consisting of FCIGRX, FCIGXL, FCIXRL, FCXGRL, FXIGRL, XCIGRL, XXIGRL, XCXGRL, XCIXRL, XCIGXL, XCIGRX, FXXGRL, FXIXRL, FXIGXL, FXIGRX, FCXXRL, FCXGXL, FCXGRX, FCIXXL, FCIXRX, and FCIGXX, wherein each X is independently a natural or synthetic amino acid residue.

47. The method of claim 43 wherein at least one of the one or more zonulin and/or ZOT receptors is a peptide comprising the sequence FCIGRL.

48. The method of claim 39 wherein the composition is in aqueous solution.

49. The method of claim 39 wherein the composition further comprises one or more protease inhibitors.

50. The method of claim 49 wherein at least one of the one or more protease inhibitors is selected from the group consisting of bestatin, L-trans-3-carboxyoxiran-2-carbonyl-L-leucylagmatine, EDTA, PMSF, aprotinin, amyloid protein precursor (APP), amyloid beta precursor protein, α1-proteinase inhibitor, collagen VI, and bovine pancreatic trypsin inhibitor (BPTI), 4-(2-aminoethyl)-benzenesulfonyl fluoride (AEBSF), antipain, benzamidine, chymostatin, ε-aminocaproate, N-ethylmaleimide, leupeptin, pepstatin A, phosphoramidon, and combinations thereof.

51. The method of claim 39 wherein the composition further comprises one or more pharmaceutically acceptable excipients.

52. The method of claim 43 wherein at least one of the one or more zonulin and/or ZOT receptors is a peptide comprising the sequence FCIGRL and the composition further comprises one or more protease inhibitors.
